(19) United States
(12) Patent Application Publication
(10) Pub. No.: US 2013/0255677 A1
(43) Pub. Date: Oct. 3, 2013

(54) DISPOSABLE RESPIRATORY CIRCUIT COUPLED WITH A DISPOSABLE TEMPERATURE SENSOR

(71) Applicant: Christopher M. Varga, Laguna Hills, CA (US)

(72) Inventor: Christopher M. Varga, Laguna Hills, CA (US)

(21) Appl. No.: 13/436,686

(22) Filed: Mar. 30, 2012

(51) Int. Cl. A61M 16/16

(52) U.S. Cl. 128/203.12

(57) ABSTRACT

A disposable respiratory gas circuit is provided. The disposable respiratory gas circuit, according to one embodiment, includes at least one disposable temperature sensor and a communication mechanism. The at least one disposable temperature sensor is coupled with the disposable respiratory gas circuit. The communications mechanism provides communication between the at least one disposable temperature sensor and a temperature monitoring system.

### FIELD OF THE INVENTION

The present technology relates generally to respiratory circuits. More particularly, the present technology relates to a disposable respiratory gas circuit.

### BACKGROUND

Humidified respiratory gas delivery for patients involves measuring, monitoring and controlling the temperature of the gases which are delivered to the patient. A respiratory gas circuit is used to deliver the gases to the patient from a humidification system or a ventilation device (also referred to herein as “gas delivery system”). A reusable removable temperature sensor is temporarily attached to the respiratory gas circuit. When the patient's treatment has completed, the temperature sensor can be removed from the respiratory gas circuit, cleaned and then reused for a different patient.

### DRAWINGS

FIGS. 1A and 1B depict disposable respiratory gas circuits using wired temperature communications, according to one embodiment.

FIGS. 2A and 2B depict disposable respiratory gas circuits using wireless temperature communications, according to one embodiment.

FIG. 3 depicts a system that includes a disposable respiratory gas circuit connected to a respiratory gas delivery system and a temperature monitoring system, according to one embodiment.

FIGS. 4A-4E depict various ways of coupling a temperature sensor wire, according to various embodiments.

FIGS. 5A-5D depict various relationships between the temperature wire and the heater wire, according to various embodiments.

FIGS. 6A-6D depict cross sections of the respiratory gas circuit wall or patient piece's wall and various locations for placing a disposable temperature sensor with respect to the respiratory gas circuit's wall or patient piece's wall (also referred to as the “patient piece wall”), according to various embodiments.

The drawings referred to in this description should not be understood as being drawn to scale unless specifically noted.

### DESCRIPTION OF EMBODIMENTS

Conventional reusable removable temperature sensors that are temporarily attached to a respiratory gas circuit, for example, using over molding involve hospital personnel manually attaching the conventional temperature sensors to the respiratory gas circuit, manually managing and draping wires alongside the respiratory gas circuit, manually removing the conventional temperature sensors when the treatment has completed, and manually cleaning the conventional temperature sensors, among other things. Thus, conventional temperature sensors result in wasted time, expense, errors due to manual effort, errors due to wires being tangled, and patient discomfort, among other things.

Therefore, according to various embodiments, a disposable respiratory gas circuit is provided that includes at least one disposable temperature sensor and a communications mechanism. The at least one disposable temperature sensor can be coupled with the disposable respiratory gas circuit. The at least one disposable temperature sensor may be incorporated into the disposable respiratory gas circuit, for example, at the time that the disposable respiratory gas circuit is manufactured or prior to arriving at a facility that uses the disposable respiratory gas circuit for a patient, among other things. The communications mechanism provides communication between the at least one disposable temperature sensor and a temperature monitoring system. The communications mechanism may be wired or wireless, as will become more evident.

Providing one or more disposable temperature sensors that are coupled to the disposable respiratory gas circuit saves time and money due to the reduction or elimination of the manual effort on the part of hospital personnel, among other things. Further, providing an at least one disposable temperature sensor that is coupled to the disposable respiratory gas circuit simplifies setup, reduces time required to begin humidified respiratory therapy, reduces the possibility of errors due to manual effort, errors due to wires getting tangled, errors due to improper assembly, and patient discomfort, among other things.

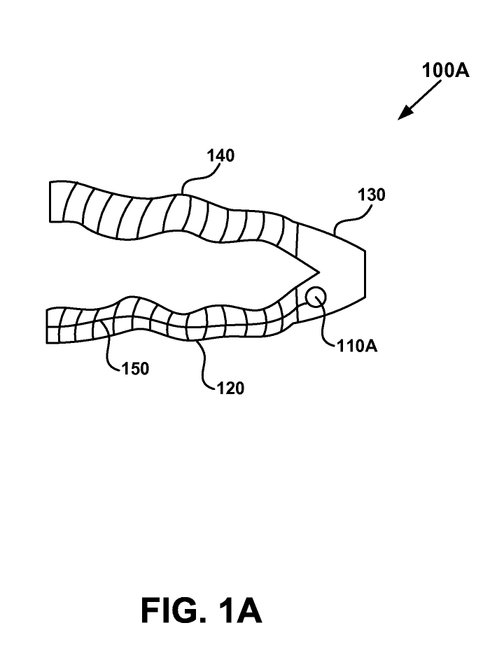

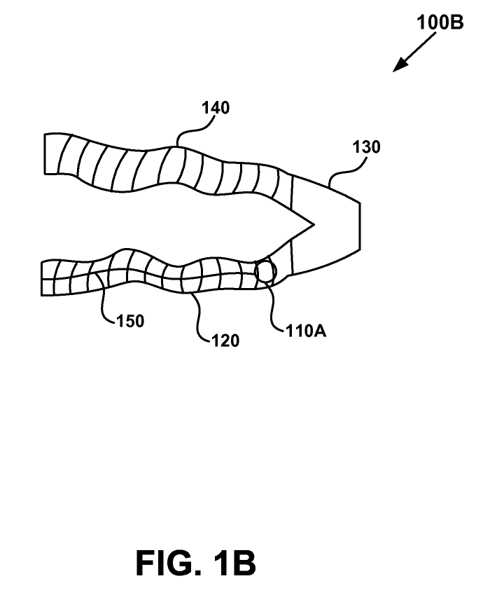

FIG. 1A depicts a disposable respiratory gas circuit 100A using wired temperature communications, according to one embodiment. The disposable respiratory gas circuit 100A includes an inspiratory limb 120, an expiratory limb 140, a patient piece 130, a disposable temperature sensor 110A and a wire 150. The inspiratory limb 120 transfers gas from a respiratory gas delivery system to the patient for inhalation. The expiratory limb 140 is optional and can be used for removing exhaled gases from the patient. The expiratory limb 140 may also be attached to a system, such as the respiratory gas delivery system, as a part of measuring and monitoring gases, as will become more evident. Additional disposable temperature sensors 110A and wires 150 may be included in the disposable respiratory gas circuit 100A.

FIG. 1B depicts a disposable respiratory gas circuit 100B using wired temperature communications, according to one embodiment. As depicted in FIG. 1B, the disposable temperature sensor 110A is located at the end of the inspiratory limb 120.

Referring to FIGS. 1A and 1B, the patient piece 130 is used to interface with the patient. An example of a patient piece 130 is a WYE which typically connects to an endotracheal interface or a tracheostomy interface. According to one embodiment, the disposable respiratory gas circuit 100A, 100B is a Y-shaped setup that has two respiratory gas limbs 120 and 140 joined by the patient piece 130. However, as stated, the expiratory limb 140 is optional. In this case, the disposable respiratory gas circuit 100A, 100B may include a single tube. The patient piece 130 may also be a mask or any other patient interface commonly used in respiratory therapy. The patient piece 130 may also be constructed of multiple parts that make up the overall interface to the patient. The patient piece 130 may include connectors. The wire 150 can be used for communicating between the disposable temperature sensor 110A and a temperature monitoring system. The temperature monitoring system can measure, monitor, or control temperature, or a combination thereof.

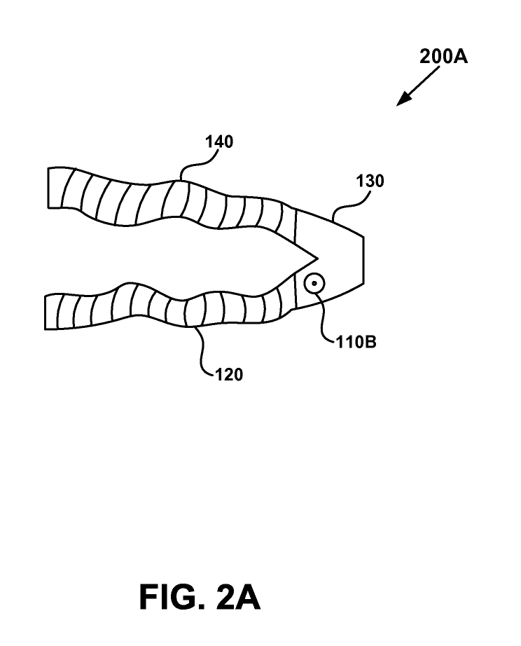

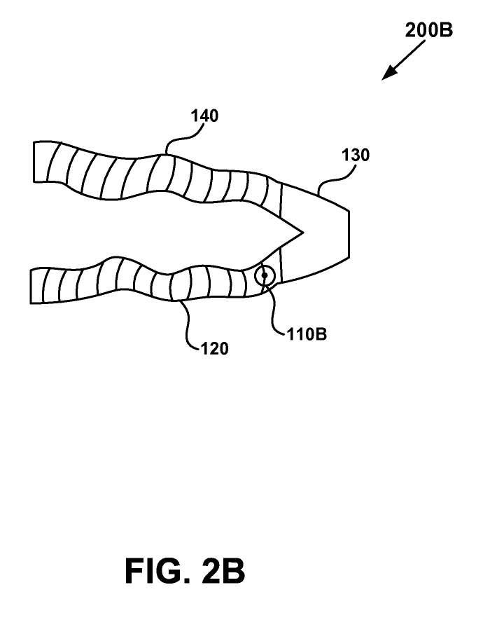

FIG. 2A depicts a disposable respiratory gas circuit 200A using wireless temperature communications, according to one embodiment. The disposable respiratory gas circuit 200A includes an inspiratory limb 120, an expiratory limb 140, a patient piece 130, and a disposable temperature sensor 110B that is capable of wireless communications. The inspiratory limb 120 transfers gas from a respiratory gas delivery system to the patient for inhalation. The expiratory limb 140 is optional and can be used for removing exhaled gases from the patient. The expiratory limb 140 may also be attached to a system, such as the respiratory gas delivery system, as a part of measuring and monitoring gases. Additional disposable temperature sensors 110B may be included in the disposable respiratory gas circuit 200A.

FIG. 2B depicts a disposable respiratory gas circuit 200B using wireless temperature communications, according to one embodiment. As depicted in FIG. 2B, the disposable temperature sensor 110B is located at the end of the inspiratory limb 120.

Referring to FIGS. 2A and 2B, the patient piece 130 is used to interface with the patient. An example of a patient piece 130 is a WYE which typically connects to an endotracheal interface or a tracheostomy interface. According to one embodiment, the disposable respiratory gas circuit 200A, 200B is a Y-shaped setup that has two respiratory gas limbs 120 and 140 joined by the patient piece 130. However, as stated, the expiratory limb 140 is optional. In this case, the disposable respiratory gas circuit 200A, 200B may include a single tube. The patient piece 130 may also be a mask or any other patient interface commonly used in respiratory therapy. The patient piece 130 may also be constructed of multiple parts that make up the overall interface to the patient. The patient piece 130 may include connectors. The disposable temperature sensor 110B, according to one embodiment, can communicate wirelessly with a temperature monitoring system. A wireless communications protocol, such as Blue Tooth, can be used. The temperature monitoring system can measure, monitor, or control temperature, or a combination thereof.

With reference to FIGS. 1A and 2A, the disposable temperature sensor 110, according to one embodiment, is located at the patient piece 130. The disposable temperature sensor 110 can be coupled with the patient piece 130. For example, the disposable temperature sensor 110 may be located in the gas path inside of the patient piece 130, inside the wall of the patient piece 130, on the inner surface of the patient piece 130's wall, or on the outer surface of the patient piece 130's wall, among other things. Further with reference to FIGS. 1A and 2A, the disposable temperature sensor 110, according to one embodiment, is located on the inspiratory limb side of the patient piece 130.

With reference to FIGS. 1B and 2B, the disposable temperature sensor 110, according to one embodiment, is located at the inspiratory limb 120. For example, the disposable temperature sensor 110 may be located in the gas path inside the inspiratory limb 120, inside the wall of the inspiratory limb 120, on the inner surface of the inspiratory limb 120's wall, or on the outer surface of the inspiratory limb 120's wall, among other things.

Although various embodiments are described and illustrated with the disposable temperature sensor 110 coupled with the inspiratory limb 120 or the patient piece 130, various embodiments are also well suited for coupling the disposable temperature sensor 110 with the expiratory limb 140. For example, the disposable temperature sensor 110 may be located in the gas path inside of the expiratory limb 140, inside the wall of the expiratory limb 140, on the inner surface of the expiratory limb 140's wall, or on the outer surface of the expiratory limb 140's wall. The disposable temperature sensor 110 may also be located at an outlet of a humidification system, among other things. Various embodiments are well suited for coupling a sensor 110 to the disposable respiratory gas circuit at a number of locations. For example, a connector can be used to couple a sensor 110 between a limb 120, 140 and a patient piece 130. A connector can be used for coupling a sensor 110 at other locations.

Although various embodiments are described and illustrated with a single disposable temperature sensor 110 coupled with a disposable respiratory circuit, various embodiments are also well suited for coupling multiple disposable temperature sensors 110 with disposable respiratory circuits 100A, 100B, 200A, 200B. For example, a disposable respiratory circuit may contain a disposable temperature sensor at each end of its inspiratory limb 120 in order to measure and monitor temperature at both the outlet of a humidifier 350 and the end of the limb 120 closest to the patient.

There are various methods of coupling the disposable temperature sensor 110 with the patient piece 130, with one or more limbs 120, 140, or with an outlet of a humidification system. For example, the disposable temperature sensor 110 may be permanently or temporarily coupled with the patient piece 130, the limbs 120 and/or 140 or an outlet of a humidification system, as will become more evident. The disposable temperature sensor 110 can be incorporated into the patient piece 130, the limbs 120 and/or 140, or the outlet of a humidification system. The disposable temperature sensor 110 can be a permanent part of the patient piece 130, the limb 120 or the limb 140, or the outlet of a humidification system, among other things.

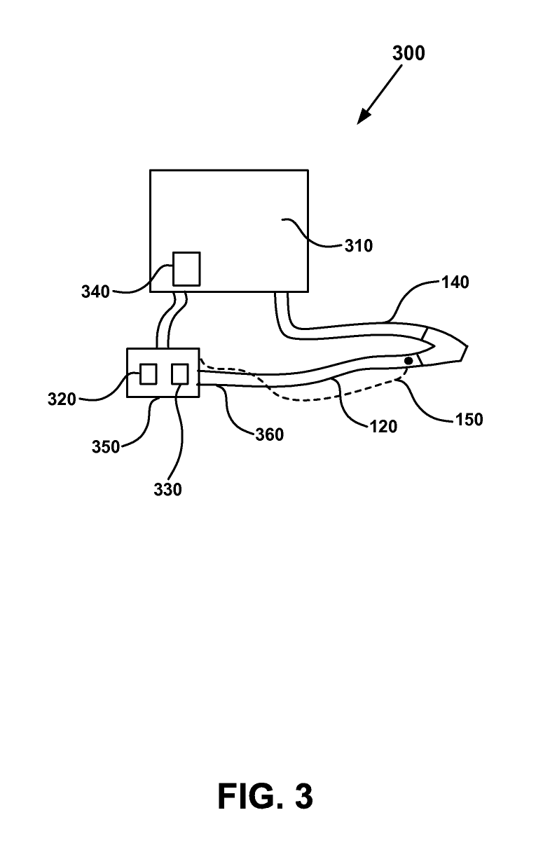

FIG. 3 depicts a system 300 that includes a disposable respiratory gas circuit 100A connected to a respiratory gas delivery system 310, according to one embodiment. An example of a respiratory gas delivery system 310 is a ventilation device 340. The temperature monitoring system 320 may be part of a humidification system 330 or may be integrated into the respiratory gas delivery system 310 or may stand alone.

The temperature monitoring system 320, according to one embodiment, monitors the temperature that is sensed by the disposable temperature sensor 110A. The temperature monitoring system 320 may also measure or control the temperature of the gas provided to the patient, or a combination there of. As depicted in FIG. 3, the temperature monitoring system 320 is a part of the humidification system 330. However, the temperature monitoring system 320 may be part of the respiratory gas delivery system 310 or may be located separately from both the humidification system 330 and the respiratory gas delivery system 310.

The gas from the ventilator 340 can be sent to the humidification system 350 where it is heated and humidified. After the gas leaves the humidification system 330, it can travel down the inspiratory limb 120 to the patient piece 130. The one or more heater wires associated with the circuit limbs 120, 140 can be used to maintain the gas temperature and prevent condensation. Gases that the patient exhales can travel back down the expiratory limb 140 and return to the ventilator 340. A single limb circuit that includes an inspiratory limb 120 can be used, for example, without an expiratory limb.

The system 300 can be used with any type of disposable respiratory gas circuit 100A, 100B, 200A, 200B (FIGS. 1A-2B), according to various embodiments. For example, although FIG. 3 depicts a system 300 that uses wired communications between a disposable temperature sensor 110 and a temperature monitoring system 320, the system 300 is well suited for communicating wirelessly between a disposable temperature sensor 110 and a temperature monitoring system 320. A wireless communications protocol, such as Blue Tooth, can be used.

According to one embodiment, a disposable temperature sensor 110 can be located near the outlet 360 of the humidifier 350 which is part of the humidification system 330. For example, a second disposable temperature sensor 110 could be located near the outlet 360. According to various embodiments, a disposable temperature sensor 110 can be located in the patient piece 130 or anywhere along the limbs 120, 140.

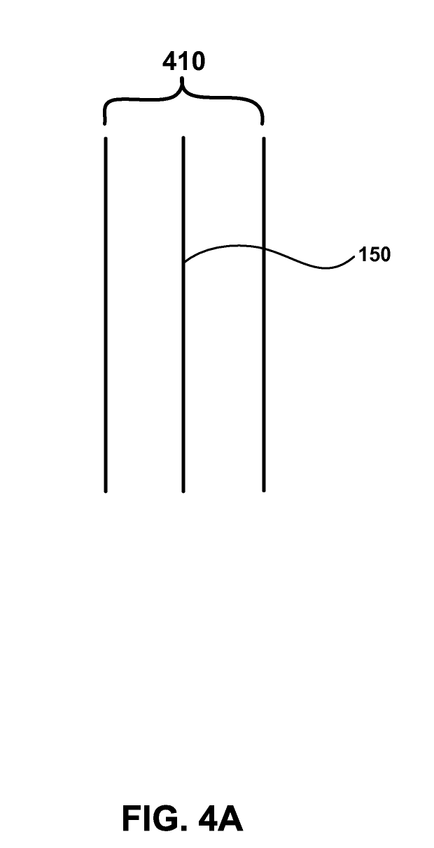

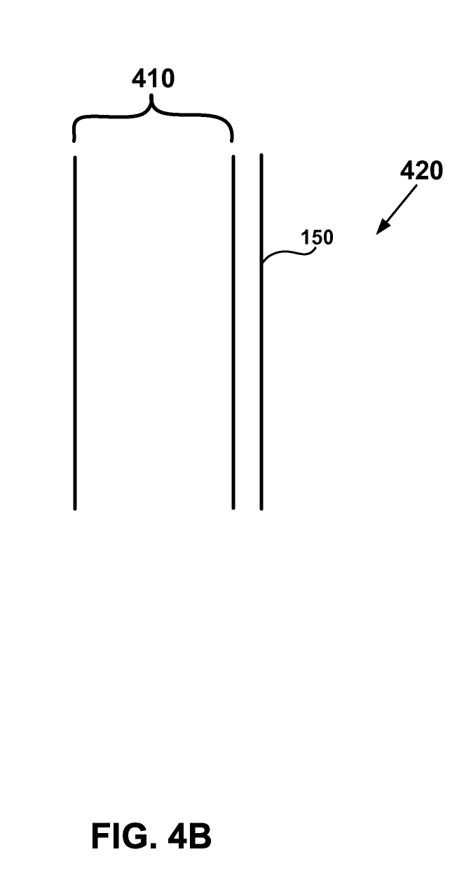

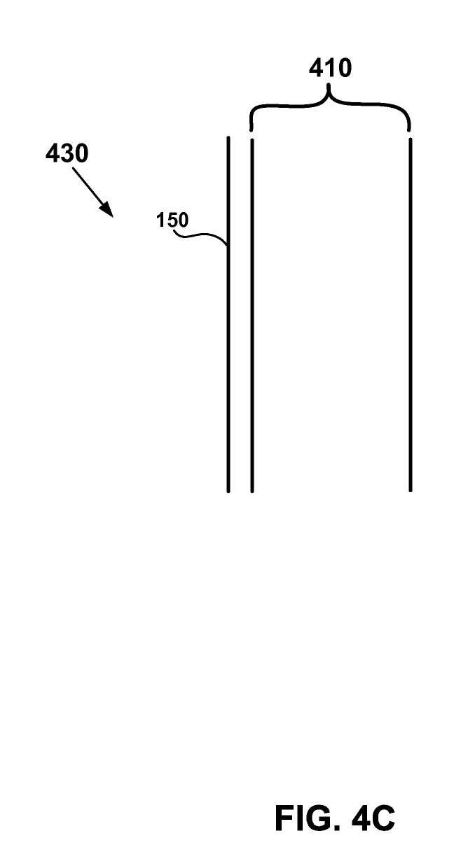

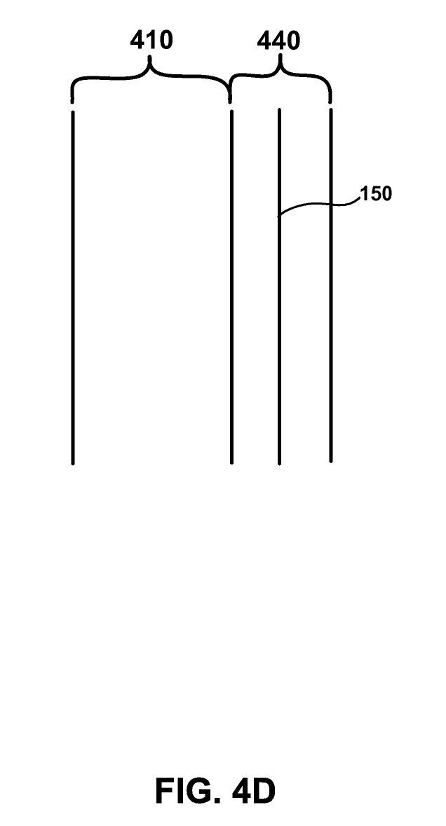

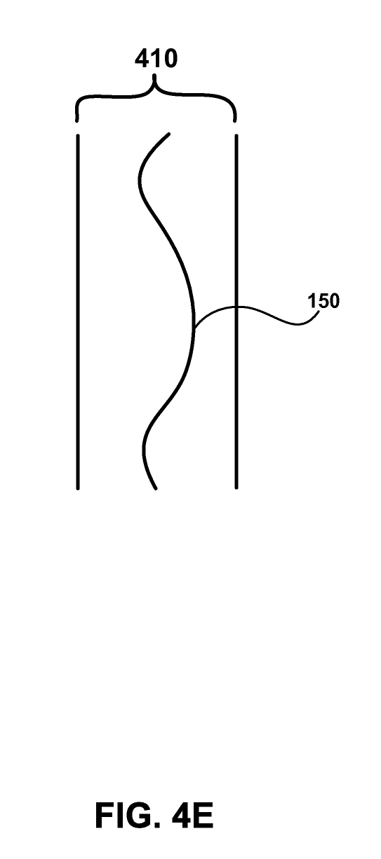

FIGS. 4A-4E depict various ways of coupling a temperature wire 150, according to various embodiments. For example, referring to FIG. 4A, the wire 150 may be incorporated inside of the wall 410 of the disposable respiratory gas system 100A, 100B, 200A, 200B (FIGS. 1A-2B). In this case, the wire 150 may be incorporated in the materials of the disposable respiratory gas system 100A, 100B, 200A, 200B's wall 410. In another embodiment, the wire 150 may run along the inner surface 420 of the disposable respiratory gas system 100A, 100B, 200A, 200B's wall 410, as depicted in FIG. 4B, or run along the outer surface 430 of the disposable respiratory gas system 100A, 100B, 200A, 200B's wall 410, as depicted in FIG. 4C. Referring to FIGS. 4B and 4C, the wire 150 may be attached to a surface of the wall 410. FIG. 4D depicts the temperature wire 150 inside a dedicated channel 440 molded into the wall 410. According to one embodiment, the temperature wire 150 can lay inside or be wound inside of one or more limbs 120, 140, in a similar manner that a heater wire is placed inside of a respiratory circuit. FIG. 4E depicts the temperature wire 150 lying inside of one or more of the limbs 120, 140.

According to one embodiment, a temperature wire 150 can be co-extruded with one or more circuit limbs 120, 140, for example, during the manufacturing process. The wire 150 may be temporarily or permanently attached to the disposable respiratory gas circuit 100A, 200A.

The phrases “the wall of the disposable temperature sensor system” or “the disposable temperature sensor system's wall” are intended to refer to the walls of either of the limbs 120, 140 (FIGS. 1A and 2A) or the wall of the patient piece 130 (FIGS. 1A and 2A), among other things.

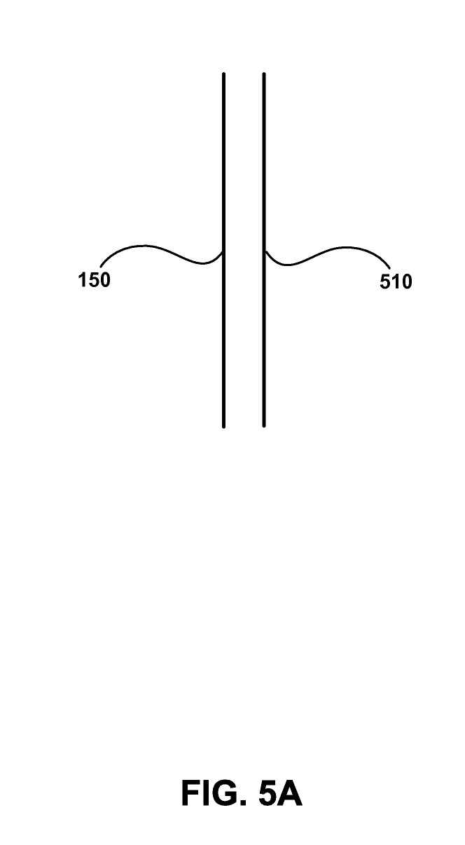

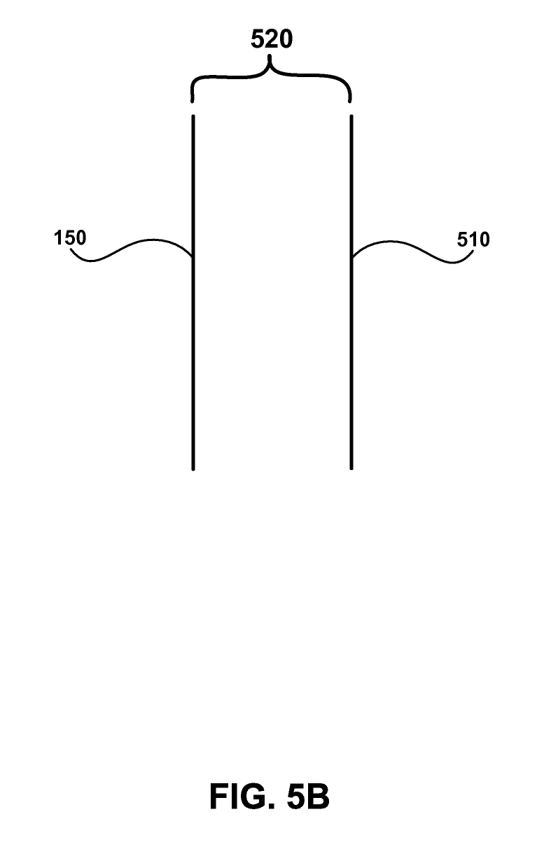

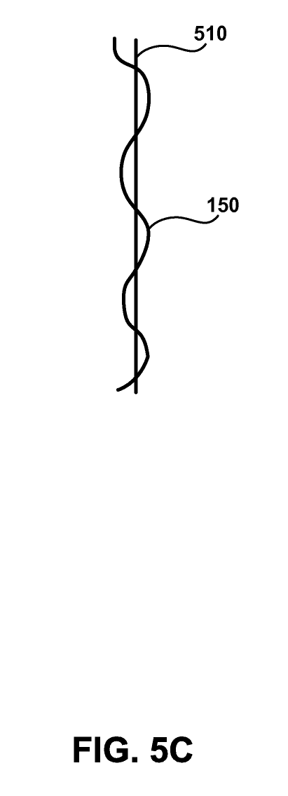

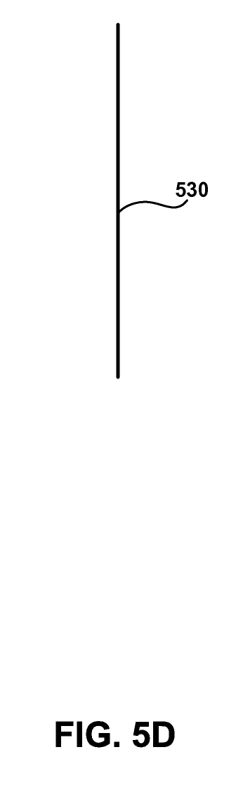

FIGS. 5A-5D depict various relationships between the temperature wire 150 and the heater wire 510, according to various embodiments. The heater wire 510 can be used to maintain the temperature of the heated and humidified respiratory gases as they travel from the humidifier to the patient. A heater wire 510 can be used to control the temperature of the gases when they reach the patient and also to prevent water from condensing out in either of the limbs due to temperature reductions. For these purposes, one or more heater wires 510 can be associated with either or both limbs.

FIG. 5A depicts the temperature wire 150 in close proximity to the heater wire 510, according to one embodiment. FIG. 5B depicts the temperature wire 150 and the heater wire 510 separated by a gap 520. For example, one of the wires 150, 510 may run along one side of a limb and the other wire 150, 510 may run along the other side of the limb. In another example, one of the wires 150, 510 may run along the bottom of a limb and another wire 150, 510 may run along the top of the limb. In yet another example, one wire 150, 510 may run along one of the limbs and the other wire 150, 510 may run along the other limb. These are just a few examples of how the wires 150, 510 may be separated by a gap 520. FIG. 5C depicts winding the temperature wire 150 around the heater wire 510, according to one embodiment. FIG. 5D depicts using a single wire 530 for both heating and temperature (referred to as a “heater temperature wire”), according to one embodiment.

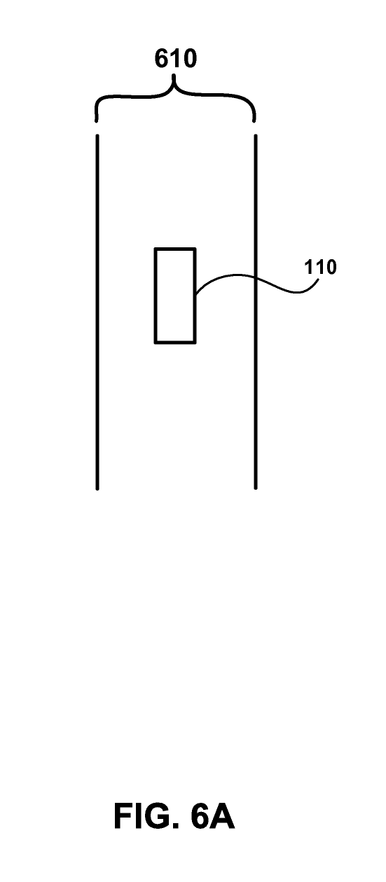

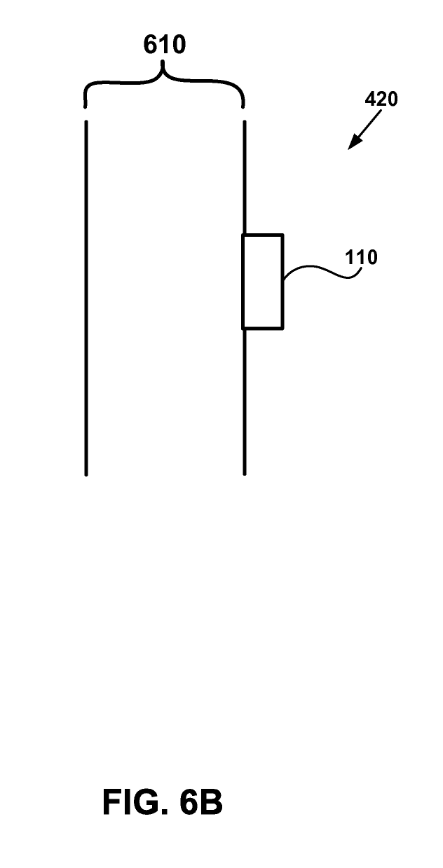

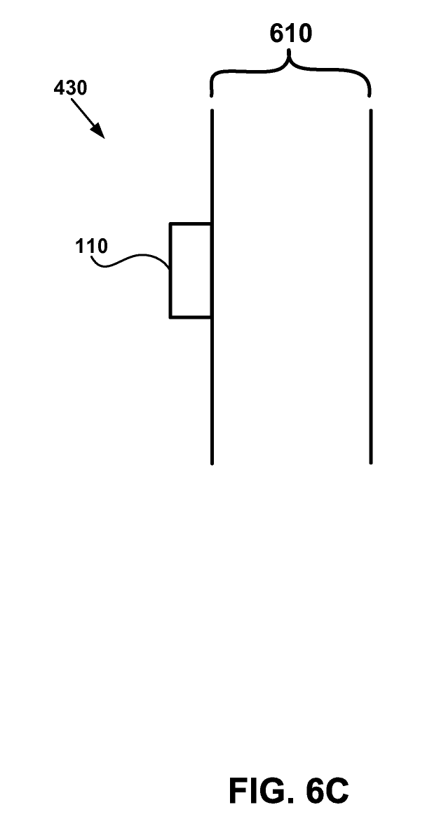

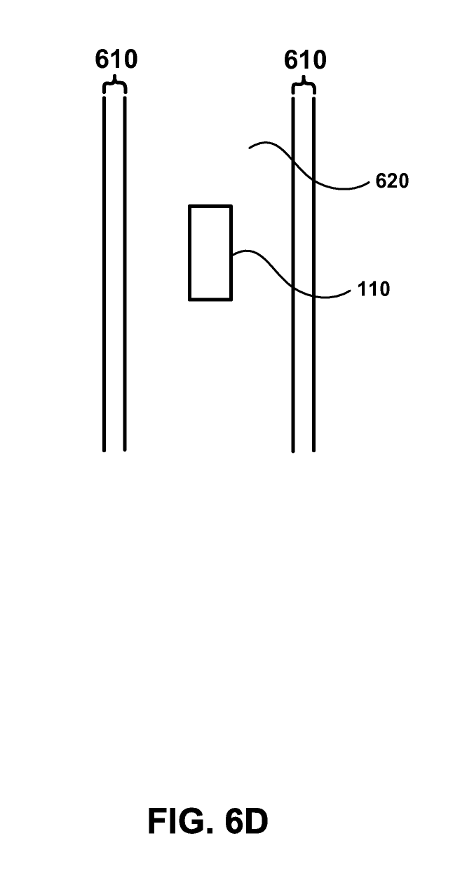

FIGS. 6A-6C depict cross sections of a wall 610 and various locations for placing a disposable temperature sensor 110 (FIGS. 1A and 2A) with respect to the wall 610, according to various embodiments. The wall 610 could be the wall of the patient piece (also referred to herein as “the patient piece wall”), the wall of either of the limbs, or the wall of the humidification system's outlet, among other things.

FIG. 6A depicts the disposable temperature sensor 110 located inside of the wall 610, according to one embodiment. For example, the disposable sensor 110 may be incorporated in the materials of the patient piece or the materials of either of the limbs. FIG. 6B depicts the disposable temperature sensor 110 located on the inner surface 420 of the wall 610, according to one embodiment. FIG. 6C depicts the disposable temperature sensor 110 located on the outer surface 430 of the wall 610, according to one embodiment. The temperature sensor 110 may also be located within the gas path inside of the limbs, within the gas path inside of the patient piece, or within the gas path inside of the outlet of the humidifier. FIG. 6D depicts the disposable temperature sensor 110 located within the respiratory gas path 620 inside of either of the limbs 120 and 140, the patient piece 130 or the outlet 360 of the humidifier 350.

The disposable temperature sensor 110 (FIGS. 1A-2B) may be attached permanently. The disposable temperature sensor 110 may be attached permanently by incorporating the disposable temperature sensor 110 inside of the wall 610, for example, as depicted in FIG. 6A. Alternatively, the disposable temperature sensor 110 may be attached temporarily to the wall 610.

There are various points in time and locations at which a disposable temporary sensor 110 (FIGS. 1A-2B) can be coupled with the disposable respiratory gas circuit 100A, 100B, 200A, 200B (FIGS. 1A-2B). For example, the coupling can occur at the time that the disposable respiratory gas circuit 100A, 100B, 200A, 200B is manufactured, prior to arriving at a facility that uses the disposable respiratory gas circuit 100A, 100B, 200A, 200B for a patient, or after arriving at the facility that uses the disposable respiratory gas circuit 100A, 100B, 200A, 200B, among other things. The coupling of the sensor 110 to the circuit can be performed by manufacturing personnel, by hospital personnel or a third party that buys components to create a circuit with a disposable temperature sensor 110, among others.

The disposable temperature sensor 110 (FIGS. 1A-2B) may be attached to the disposable respiratory gas circuit 100A, 100B, 200A, 200B (FIGS. 1A-2B) by the hospital personnel. However, according to various embodiments, the hospital personnel are not required to detach the disposable temperature sensor 110, clean disposable temperature sensor 110, and reattach the disposable temperature sensor 110 to another respiratory gas circuit 100A, 100B, 200A, 200B since the sensor 110 is disposable and, therefore, can be disposed of with the circuit. According to one embodiment, the disposable respiratory gas circuit 100A, 100B, 200A, 200B with the coupled disposable temperature sensor 110 is for single patient use. For example, a disposable respiratory gas circuit 100A, 100B, 200A, 200B can be disposed of after a patient has used it.

There has been a long felt need for reducing the wasted time, expense, errors due to manual effort, errors due to wires being tangled, and patient discomfort, among other things, associated with conventional temperature sensors. However, to date, a disposable temperature sensor has not been used to reduce the wasted time, expense, errors due to manual effort, errors due to wires being tangled, and patient discomfort, among other things, associated with conventional temperature sensors.

Examples of the subject matter are thus described. Although the subject matter has been described in a language specific to structural features and/or methodological acts, it is to be understood that the subject matter defined in the appended claims is not necessarily limited to the specific features or acts described above. Rather, the specific features and acts described above are disclosed as example forms of implementing the claims.

Various embodiments have been described in various combinations. However, any two or more embodiments may be combined. Further, any embodiment may be used separately from any other embodiment. Features, structures, or characteristics of any embodiment may be combined in any suitable manner with one or more other features, structures, or characteristics.

What is claimed is:

1. A disposable respiratory gas circuit comprising:
at least one disposable temperature sensor coupled with the disposable respiratory gas circuit, and
a communications mechanism for providing communication between the at least one disposable temperature sensor and a temperature monitoring system.

2. The disposable respiratory gas circuit of claim 1, wherein the at least one disposable temperature sensor is incorporated into the disposable respiratory gas circuit.

3. The disposable respiratory gas circuit of claim 1, wherein the disposable respiratory gas circuit includes an inspiratory limb and wherein the at least one disposable temperature sensor is located at the inspiratory limb.

4. The disposable respiratory gas circuit of claim 1, wherein the disposable respiratory gas circuit includes an expiratory limb and wherein the disposable temperature sensor is located at the expiratory limb.

5. The disposable respiratory gas circuit of claim 1, wherein the disposable respiratory gas circuit includes a patient piece and wherein the at least one disposable temperature sensor is located at the patient piece.

6. The disposable respiratory gas circuit of claim 1, wherein the communication mechanism is selected from a group consisting of wired and wireless.

7. The disposable respiratory gas circuit of claim 1, wherein the disposable respiratory gas circuit does not require a temperature wire.

8. The disposable respiratory circuit of claim 1, wherein the disposable respiratory gas circuit is configured for coupling with a respiratory gas delivery system.

9. The disposable respiratory circuit of claim 1, wherein the disposable respiratory gas circuit includes an at least one temperature wire that is positioned at a location selected from a group consisting of inside of a wall of the disposable respiratory gas circuit, on an inner surface of the wall of the disposable respiratory gas circuit, on an outer surface of the wall of the disposable respiratory gas circuit, inside a dedicated channel in the wall of the disposable respiratory gas circuit, and inside a respiratory gas path formed by the wall of the disposable respiratory gas circuit.

10. The disposable respiratory gas circuit of claim 1, wherein the disposable respiratory gas circuit includes an at least one temperature wire that has a relationship to a heater wire that is selected from a group consisting of close proximity between the at least one temperature wire and the heater wire, not in close proximity between the at least one temperature wire and the heater wire, the at least one temperature wire is wound around the heater wire, and a single wire for temperature sensing and heating.

11. The disposable respiratory gas circuit of claim 1, wherein the at least one disposable temperature sensor is positioned at a location selected from a group consisting of inside of a wall of the disposable respiratory gas circuit, on an inner surface of the disposable respiratory gas circuit's wall, on an outer surface of the disposable respiratory gas circuit's wall, and inside a respiratory gas path formed by the wall of the disposable respiratory gas circuit.

12. The disposable respiratory gas circuit of claim 1, wherein the disposable respiratory gas circuit includes a patient piece, and wherein the at least one disposable temperature sensor is positioned at a location selected from a group consisting of inside of a wall of the patient piece, on an inner surface of the wall of the patient piece, on an outer surface of the wall of the patient piece, and inside of a respiratory gas path formed by the wall of the patient piece.

13. A disposable respiratory gas circuit comprising:
an at least one disposable temperature sensor that does not require hospital personnel to manually attach the at least one disposable temperature sensor to the disposable respiratory gas circuit; and
a communications mechanism for providing communication between the at least one disposable temperature sensor and a temperature monitoring system.

14. The disposable respiratory gas circuit of claim 13, wherein the at least one disposable temperature sensor is incorporated inside of a wall of the disposable respiratory gas circuit.

15. The disposable respiratory gas circuit of claim 13, wherein the at least one disposable temperature sensor is permanently coupled to the disposable respiratory gas circuit.

16. The disposable respiratory gas circuit of claim 13, wherein the at least one disposable temperature sensor is temporarily coupled to the disposable respiratory gas circuit.

17. The disposable respiratory gas circuit of claim 13, wherein the at least one disposable temperature sensor does not require any one or more of manual removal, manual cleaning, and manual reattachment of the at least one disposable temperature sensor to a different respiratory gas circuit.

18. The disposable respiratory gas circuit of claim 13, wherein the communication mechanism is selected from a group consisting of wired and wireless.

19. A disposable respiratory gas circuit comprising:
an at least one disposable temperature sensor that is coupled with the disposable respiratory gas circuit, wherein the coupling occurs prior to arriving at a facility that uses the disposable respiratory gas circuit for a patient; and
a communications mechanism for providing communication between the at least one disposable temperature sensor and a temperature monitoring system.

20. The disposable respiratory gas circuit of claim 19, wherein the at least one disposable temperature sensor is incorporated into the disposable respiratory gas circuit.

21. The disposable respiratory gas circuit of claim 19, wherein the disposable respiratory gas circuit includes an inspiratory limb and wherein the at least one disposable temperature sensor is located at the inspiratory limb.

22. The disposable respiratory gas circuit of claim 19, wherein the communication mechanism is selected from a group consisting of wired and wireless.

23. The disposable respiratory circuit of claim 19, wherein the disposable respiratory gas circuit includes an at least one temperature wire that is positioned at a location selected from a group consisting of inside of a wall of the disposable respiratory gas circuit, on an inner surface of the wall of the disposable respiratory gas circuit, on an outer surface of the wall of the disposable respiratory gas circuit, and inside of a respiratory gas path formed by the wall of the disposable respiratory gas circuit.

24. The disposable respiratory gas circuit of claim 19, wherein the at least one disposable temperature sensor is positioned at a location selected from a group consisting of inside of a wall of the disposable respiratory gas circuit, on an inner surface of the wall of the disposable respiratory gas circuit, on an outer surface of the wall of the disposable respiratory gas circuit, and inside of a respiratory gas path formed by the wall of the disposable respiratory gas circuit.
